(19) United States
(12) Patent Application Publication
(10) Pub. No.: US 2008/0269883 A1
(43) Pub. Date: Oct. 30, 2008

(54) OCULAR IMPLANT TO CORRECT DYSPHOTOPSIA, GLARE, HALOS AND DARK SHADOW TYPE PHENOMENA

(75) Inventors: Kamel K. Das, Arlington, TX (US); Drew Morgan, Fort Worth, TX (US)

Correspondence Address: ALCON, IP LEGAL, TB4-8, 6201 SOUTH FREEWAY, FORT WORTH, TX, 76134, US

(73) Assignee: ALCON, INC., Fort Worth, TX (US)

(21) Appl. No.: 11/742,320

(22) Filed: Apr. 30, 2007

(51) Int. Cl. A61F 2/16

(52) U.S. Cl. 623/6.17

(57) ABSTRACT

Methods and devices for inhibiting the dark shadow effect, known as dysphotopsia, perceived by some subjects having implanted intraocular lenses (IOLs) are presented. In one aspect, an IOL can include an optic and one or more fixation members for facilitating placement of the IOL. The fixation member can be adapted to position the optic sufficiently close to the iris to inhibit dysphotopsia. As some examples, a fixation member can position an optic to within some distance of the tip of the iris, or the fixation member can be adapted to contact a portion of an eye posterior to an optic's posterior surface; or the fixation member can have an end that is posterior to a posterior surface of the optic. Various techniques for achieving these improvements among others are discussed, both in terms of the structures of improved IOLs, and methods for alleviating dysphotopsia.

### CROSS REFERENCE TO RELATED APPLICATIONS

This application is related to the following patent applications that are concurrently filed herewith: “Intraocular Lens with Asymmetric Haptics” (Attorney Docket No. 3227); “Intraocular Lens with Asymmetric Optics” (Attorney Docket No. 3360); “Intraocular Lens with Peripheral Region Designed to Reduce Negative Dysphotopsia” (Attorney Docket No. 2817); “IOL Peripheral Surface Designs To Reduce Negative Dysphotopsia” (Attorney Docket No. 3345); “Product Solutions to Reduce Negative Dysphotopsia” (Attorney Docket No. 3225); “Graduated Blue Filtering Intraocular Lens” (Attorney Docket No. 2962); and “Haptic Junction Designs to Reduce Negative Dysphotopsia” (Attorney Docket No. 3344), each of which is incorporated herein by reference.

### FIELD OF THE INVENTION

The present invention relates generally to intraocular lenses (IOLs), and particularly to IOLs that provide a patient with an image of a field of view without the perception of dark visual artifacts in the peripheral visual field.

### BACKGROUND OF THE INVENTION

The optical power of the eye is determined by the optical power of the cornea and that of the natural crystalline lens, with the lens providing about a third of the eye's total optical power. The process of aging as well as certain diseases, such as diabetes, can cause clouding of the natural lens, a condition commonly known as cataract, which can adversely affect a patient's vision.

Intraocular lenses (IOLs) are routinely employed to replace such a clouded natural lens. Although such IOLs can substantially restore the quality of a patient's vision, some patients in whose eyes conventional IOLs are implanted occasionally report the perception of dark shadows, particularly in their temporal peripheral visual fields. This phenomenon is generally referred to as dysphotopsia.

Accordingly, there is a need for enhanced IOLs, and particularly for IOLs and methods that inhibit the perception of dark shadows in the peripheral visual field.

### SUMMARY OF THE INVENTION

The present invention is based, in part, on the discovery that the shadows perceived by IOL patients can be caused by a double imaging effect when light enters the eye at very large visual angles. More specifically, it has been discovered that in many conventional IOLs, most of the light entering the eye is focused by both the cornea and the IOL onto the retina, but some of the peripheral light misses the IOL and it is hence focused only by the cornea. This leads to the formation of a second peripheral image. Although this image can be valuable since it extends the peripheral visual field, in some IOL users it can result in the perception of a shadow-like phenomenon that can be distracting.

To reduce the potential complications of cataract surgery, designers of modern IOLs have sought to make the optical component (the “optic”) smaller (and preferably foldable) so that it can be inserted into the capsular bag with greater ease following the removal of the patient's natural crystalline lens. The reduced lens diameter, and foldable lens materials, are important factors in the success of modern IOL surgery, since they reduce the size of the corneal incision that is required. This in turn results in a reduction in corneal aberrations from the surgical incision, since often no suturing is required. The use of self-sealing incisions results in rapid rehabilitation and further reductions in induced aberrations. However, a consequence of the optic diameter choice is that the IOL optic may not always be large enough (or may be too far displaced from the iris) to receive all of the light entering the eye.

Moreover, the use of enhanced polymeric materials and other advances in IOL technology have led to a substantial reduction in capsular opacification, which has historically occurred after the implantation of an IOL in the eye, e.g., due to cell growth. Surgical techniques have also improved along with the lens designs, and biological material that previously affected light near the edge of an IOL, and in the region surrounding the IOL, no longer does so. These improvements have resulted in a better peripheral vision, as well as a better foveal vision, for the IOL users. Though a peripheral image is not seen as sharply as a central (axial) image, peripheral vision can be very valuable. For example, peripheral vision can alert IOL users to the presence of an object in their field of view, in response to which they can turn to obtain a sharper image of the object. It is interesting to note in this regard that the retina is a highly curved optical sensor, and hence can potentially provide better off-axis detection capabilities than comparable flat photosensors. In fact, though not widely appreciated, peripheral retinal sensors for visual angles greater than about 60 degrees are located in the anterior portion of the eye, and are generally oriented toward the rear of the eye. In some IOL users, however, the enhanced peripheral vision can lead to, or exacerbate, the perception of peripheral visual artifacts, e.g., in the form of shadows.

Dysphotopsia (e.g., negative dysphotopsia) is often observed by patients in only a portion of their field of vision because the nose, cheek and brow block most high angle peripheral light rays—except those entering the eye from the temporal direction. Moreover, because the IOL is typically designed to be affixed by haptics to the interior of the capsular bag, errors in fixation or any asymmetry in the bag itself can exacerbate the problem—especially if the misalignment causes more peripheral temporal light to bypass the IOL optic.

The present invention generally provides intraocular lenses (IOLs) and methods of vision correction that utilize them, which can alleviate, and preferably eliminate, the perception of dark shadows that some IOL patients occasionally report. Such IOLs can be implanted posterior or anterior to the iris of the eye. In some aspects of the present invention, the fixation members of an IOL are adapted so as to project the IOL's optic toward the iris in order to alleviate dysphotopsia. For example, an optic can be positioned sufficiently close to the iris of the eye to receive peripheral light rays entering the eye (e.g., at visual angles in a range of about 50 degrees to about 80 degrees) and to direct those rays onto the retina so as to inhibit the formation of a secondary peripheral image or to cause a reduction of the shadow region between such a secondary image and an image formed by the IOL. For example, a fixation member can extend posteriorly from the optic to project the optic toward the iris when the IOL is appropriately implanted. In some cases, the fixation member can have arm-like extensions that extend posteriorly from the optic and form an angle relative to a principal plane of the IOL's optic, e.g., in a range of about 5 degrees to about 45 degrees, or about 15 degrees to about 30 degrees. In many embodiments, the IOLs are preferably deformable such that their delivery to a subject's eye is facilitated. These, as well as other, aspects are disclosed in more detail herein.

In one aspect, an intraocular lens (herein “IOL”) is disclosed that includes an optic suitable for implantation in the eye of a subject, as well as one or more fixation members coupled to the optic and adapted to position the optic sufficiently close to the iris to inhibit the perception of peripheral visual artifacts, e.g., dysphotopsia.

In a related aspect, in the above IOL, the fixation members can project the optic toward the iris to ensure sufficient proximity of the optic to the pupil. By way of example, one or more fixation members can be adapted to position an anterior-most portion of the IOL's optic at an axial distance less than about 0.8 mm, or less than about 0.7 mm, or less than about 0.6 mm relative to a tip of the eye's iris.

The fixation members can have a variety of shapes and configurations. For instance, a fixation member can include one or more extension members that are coupled to a peripheral portion of the IOL's optic. In a particular example, an extension member can be configured as an annular structure that is coupled to the peripheral portion of the optic. Such an annular structure can be adapted to position the optic in a capsular bag of the eye, and can optionally include one or more protuberances that extend from a surface thereof to contact the capsular bag. For example, one or more protuberances can contact either the anterior surface, the posterior surface, or both surfaces of the capsular bag. In another example, a fixation member can be in the form an arm-like extension that extends posteriorly from the optic.

Another aspect is directed to an IOL that includes an optic for implantation in the eye of a subject. The IOL can also include one or more haptics, which can be coupled to the optic. Any of the haptics can have a free-end that is positioned posterior to a posterior-surface of the optic. For example, the free-end of the haptic can be separated from the optic's posterior-surface by an axial distance of at least about 0.4 mm, or at least about 0.5 mm, or at least about 0.6 mm. The IOL can be implanted in a subject's eye such that the optic intercepts peripheral light rays entering the pupil at particular angles (e.g., from about 50 degrees to about 80 degrees relative to the eye's visual axis). For example, the fixation members can be employed to position an anterior-most portion of the optic an axial distance of less than about 0.8 mm from a tip of the iris. One of more of the haptics can also be adapted to contact a portion of the eye posterior to an anterior-most portion of the optic.

An IOL includes an optic and one or more fixation members coupled to the optic in another aspect of the invention. Any of the fixation members can be adapted to position the optic such that the anterior-most portion of the optic and a tip of iris are an axial distance of less than about 0.8 mm, or less than about 0.7 mm, or less than about 0.6 mm apart when the IOL is implanted. Any one of the fixation members can also be adapted to intercept peripheral light rays (e.g., rays entering the pupil at angles from about 50 degrees to about 80 degrees relative to the eye's visual axis), and/or to contact a portion of the eye posterior to an anterior-most portion of the IOL's optic. Any fixation member can be configured consistent with any of the earlier described fixation members. For example, a fixation member can be an extension member (e.g., an annular structure) or as a haptic.

Another aspect is directed to a method of inhibiting dysphotopsia in a patient having an implanted IOL by positioning an anterior surface of the IOL's optic close enough to the iris to inhibit dysphotopsia. For instance, the anterior surface can be positioned such that the anterior surface would intercept peripheral light rays and would direct those rays to the retina so as to inhibit the formation of a secondary image on the retina or to reduce the extent of a retinal dark (shadow) region between such a secondary image and an image formed by the optic. In many cases, such peripheral light rays can enter the eye at an angle in the range from about 50 degrees to about 80 degrees relative to the eye's visual axis. By way of example, in some cases, the optic's anterior surface can be positioned an axial distance of less than about 0.8 mm from a tip of an iris of the subject's eye.

Other aspects are directed to methods of inhibiting dysphotopsia in a patient's eye by implanting an IOL therein. The IOL can be consistent with any of the embodiments discussed herein.

### BRIEF DESCRIPTION OF THE DRAWINGS

Various features of embodiments of the present invention will be more readily understood from the following detailed description when read in conjunction with the appended drawings (not necessarily drawn to scale), in which:

FIG. 1 is a schematic cross-sectional top view of a left eyeball with an intraocular lens implanted therein;

FIG. 2A is a schematic anterior view of an IOL consistent with some embodiments of the present invention;

FIG. 2B is a schematic side cross-sectional view of the IOL depicted in FIG. 2A;

FIG. 2C is a schematic side cross-sectional view of the IOL depicted in FIGS. 2A and 2B implanted in the eye of a subject;

FIG. 3 is a schematic cross-sectional top view of the left eyeball depicted in FIG. 1 with an intraocular lens consistent with some embodiments of the invention;

FIG. 4A is a schematic anterior view of an IOL having four extensions consistent with an embodiment of the present invention;

FIG. 4B is a schematic side view of the IOL depicted in FIG. 4A;

FIG. 5A is a schematic anterior view of an IOL having an annular structure with protuberances consistent with some embodiments of the present invention;

FIG. 5B is a schematic side view of the IOL depicted in FIG. 5A;

FIG. 6A is a schematic anterior view of an IOL having an annular structure consistent with some embodiments of the present invention;

FIG. 6B is a schematic side view of the IOL depicted in FIG. 6A;

FIG. 6C is a schematic side cross-sectional view of the IOL depicted in FIGS. 6A and 6B implanted in the eye of a subject;

FIG. 7A is a schematic posterior view of an IOL having an annular structure and protuberances on a posterior surface of the structure consistent with some embodiments of the present invention;

FIG. 7B is a schematic side view of the IOL depicted in FIG. 7A;

FIG. 7C is a schematic side cross-sectional view of the IOL depicted in FIGS. 7A and 7B implanted in the eye of a subject;

FIG. 8A is a schematic view of a deformable IOL folded in half, consistent with some embodiments of the present invention; and

FIG. 8B is a schematic view of the deformable IOL depicted in FIG. 8A when the IOL is in a non-deformed state.

### DETAILED DESCRIPTION OF EMBODIMENTS OF THE INVENTION

The present invention generally provides intraocular lenses (IOLs) and methods for correcting vision that employ such lenses, which can ameliorate, and preferably prevent, the perception of dark shadows that some IOL patients report.

The term “intraocular lens” and its abbreviation “IOL” are used herein interchangeably to describe devices that include one or more optics (e.g., lenses) that are implanted into the interior of the eye to either replace the eye's natural lens or to otherwise augment vision regardless of whether or not the natural lens is removed. Intracorneal lenses and phakic lenses are examples of lenses that may be implanted into the eye without removal of the natural lens.

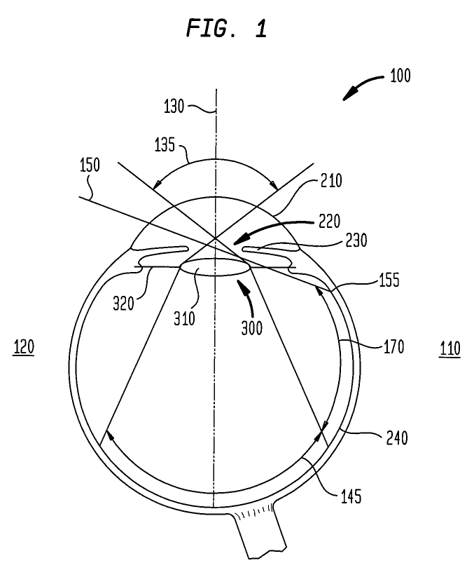

FIG. 1 presents a schematic cross-sectional top view of the left eyeball 100 of a subject having a conventional IOL 300 implanted therein. Light traveling from a field of view 135 passes through the cornea 210 and proceeds through the pupil 220 to impinge upon an optic 310 of the IOL 300. The combined optical power of the cornea and the optic focuses the light to form an image on a region 145 of the retina 240. It has been discovered that in many conventional IOLs, which can be implanted in the posterior chamber of the eye, some of the light rays entering the eye at large visual angles (e.g., depicted by an exemplary light ray 150 in FIG. 1) miss the IOL's optic 310, passing through the space between the iris 230 and the optic 310, and are hence refracted only by the cornea to be incident on a portion of the retina 155 removed from the more central imaging region 145. Such light rays, herein termed “peripheral light rays,” typically enter from the temporal direction 120 and impinge upon the nasal side 110 of the retina as shown in FIG. 1. These peripheral light rays can form a secondary image or lighted region, with a reduced intensity region 170 linking the secondary image to the more central imaging region 145. The term “secondary image” as utilized herein is not strictly limited to a focused image on the retina, though peripheral light rays typically undergo focusing upon passage through the cornea. Indeed, such “imaging” can include any type of illumination of a retinal portion removed from the more central retinal region in which an image of field of view is formed by the focusing function of both the cornea and the IOL.

Though the presence of the secondary image can potentially aid in the peripheral visual perception of a subject, the separation of the two illuminated portions of the retina can result in the perception of a shadow-like phenomenon in a region between those images. It is hypothesized that this shadow-like perception is due to the presence of a reduced intensity region 170 on the retina between a primary image 145 and a secondary image 155. This phenomenon is known as dysphotopsia, and is typically perceived on the temporal side of the subject's field of view. Dysphotopsia can also occur as a result of light reflection effects within an IOL's optic.

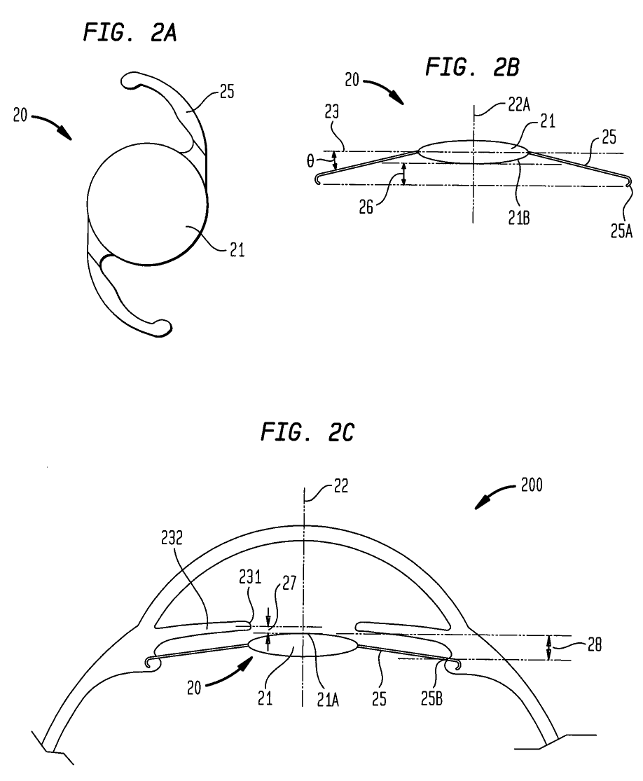

FIGS. 2A and 2B schematically present a anterior view and a side view, respectively, of an exemplary embodiment of an implantable IOL 20, which is adapted to alleviate, and preferably prevent, dysphotopsia. The IOL 20 can include an optic 21 for forming an image of a field of view on the subject's retina. Such optics can typically be implanted posterior to the iris of a subject's eye, e.g., as the lens 310 shown in FIG. 1. One or more fixation members 25 coupled to the optic 21 can be used to facilitate placement of the optic in the eye, for example, by anchoring the optic in a particular orientation. For the IOL shown in FIG. 2A, the fixation members 25 are configured as two haptics (i.e., support structures coupled to a peripheral portion of the optic) each having arm-like extensions, which can couple to a structure of the eye (e.g., a cilary body, a portion of the capsular bag, or a region between the root of the iris and the cilary body) for anchoring the optic in the eye in a desired orientation.

In this exemplary embodiment, the fixation members are adapted to position the optic sufficiently close to an eye's iris to inhibit the occurrence of dysphotopsia. For example, as shown in the side view of the IOL 20 depicted in FIGS. 2B and 2C, the fixation members 25 are posteriorly slanted relative to the optic 21 so as to project the optic 21 toward the iris 232 once the IOL 20 is implanted in the eye 200. In this embodiment, each fixation member 25 is oriented at an angle θ relative to a principal plane 23 of the optic 21, where the angle θ can be in a range of about 0 degrees to about 45 degrees. Alternatively, the angle θ can range from about 5 degrees to about 45 degrees, or from about 12 degrees to about 45 degrees, or from about 15 degrees to about 45 degrees. In further alternatives, the angle θ can range from about 5 degrees to about 30 degrees, or from about 12 degrees to about 30 degrees, or from about 15 degrees to about 30 degrees.

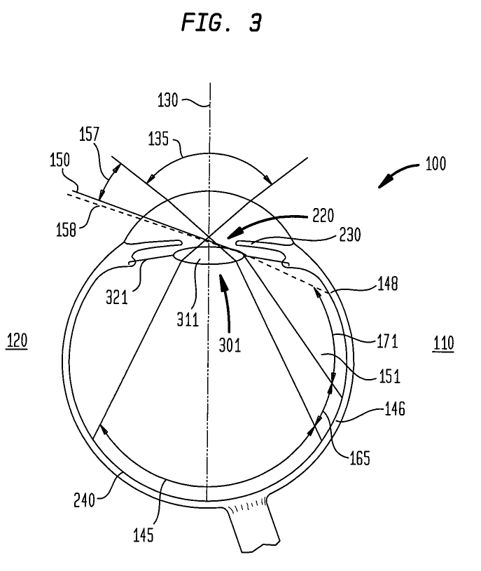

An example of how an IOL, according to an embodiment of the present invention, can alleviate dysphotopsia is provided herein with reference to FIG. 3, which schematically depicts the left eye of FIG. 1 in which an IOL 301 is implanted. The slanted haptics 321 of the IOL 301 project the IOL's optic 311 toward the iris 230 such that the optic 311 is positioned closer to the pupil 220 than the optic 310 of the conventional IOL 300 shown in FIG. 1. In this manner, the optic 311 can receive peripheral light rays that would have otherwise missed the optic 311. For example, a peripheral light ray 150, which would not impinge on the optic 310 of FIG. 1, can now be incident on the optic 311 to be directed as light ray 151 to image onto a position 146 the retina 240. In this manner, the formation of a second peripheral image that could result in dysphotopsia can be avoided.

As shown in FIG. 3, an IOL 301 can be adapted such that a range of peripheral light rays 157 entering a pupil 220 of the eye 100 (e.g., light rays entering the eye at visual angles in a range of about 50 degrees to about 80 degrees) can be intercepted by the optic 311, and can be focused onto the retina so as to form a single image of a field of view.

In some cases, even with the reduction of the axial distance between the IOL's optic and the iris, some peripheral light rays 158 entering the eye might still miss the optic 311, and hence form a secondary peripheral image 148 on the retina 240 as depicted in FIG. 3. The close proximity of the optic to the iris, however, results in an appreciably smaller dark region 171 between a secondary image 148 and an image 145, 165 formed by the optic 311. More particularly, the optic's reduced axial distance results in expansion of the peripheral portion 165 of an image of a field of view formed on the retina, due to focusing of peripheral light rays 157 entering the eye at relatively large visual angles, which can lead to a reduction in the size of the dark region 171. In some cases, allowing some peripheral light rays to miss the IOL and be focused only by the cornea onto the retina, while decreasing the size of the shadow region, can advantageously enhance the subject's peripheral vision while also alleviating or eliminating the effects of dysphotopsia. Accordingly, in some embodiments the IOL can be situated to capture a particular range of peripheral light rays, while allowing some peripheral light to miss the optic and be focused only by the cornea upon the retina. It is understood, however, that some embodiments utilizing appropriately adapted IOLs can substantially eliminate the effect of peripheral light rays missing the optic before impinging onto the retina.

Optics, as utilized by a variety of the embodiments disclosed herein, are preferably formed of a biocompatible material, such as soft acrylic, silicone, hydrogel, or other biocompatible polymeric materials having a requisite index of refraction for a particular application. For example, in some embodiments, the optic can be formed of a cross-linked copolymer of 2-phenylethyl acrylate and 2-phenylethyl methacrylate, which is commonly known as Acrysof®.

The term “fixation member” as utilized herein can refer to any structure that is coupled to the optic for positioning the IOL in a desired orientation upon implantation in a subject's eye, typically in a manner such that the optic acts as an effective optical aid to the subject. FIGS. 2A-2C, 4A, 4B, 5A, 5B, 6A-6C, and 7A-7C provide some examples of fixation members according to various embodiments of the invention, and are described in more detail herein. Similar to the optic, a fixation member can also be formed of a suitable biocompatible material, such as polymethylmethacrylate. While in some embodiments, a fixation member can be formed integrally with the optic, in other embodiments, the fixation member is formed separately and attached to the optic in a manner known in the art.

Referring back to the exemplary IOL depicted in FIGS. 2A-2C, features of the depicted IOL 20 can be utilized by various embodiments of the present invention individually or in any combination. In some embodiments, one or more fixation members of an IOL can be adapted to position the IOL's optic such that an anterior-most portion of the optic and a tip of the iris are separated by an axial distance in a given range, e.g., less than a threshold value. As shown in FIG. 2C, an anterior surface 21A of the optic 21 and the tip of the iris 231 can be separated by an axial distance 27, which is substantially parallel to an optical axis 22, which can be the optical axis of the optic or the optical axis of the eye with or without the IOL 20 implanted therein. In some instances, the optical axis of the optic 22A can be substantially coincident with the eye itself. The axial distance 27 can be chosen such that the optic would receive at least a portion of the peripheral light rays that typically bypass the optic 21 in prior art IOLs. For instance, the axial distance 27 can be sufficiently small such that the optic would receive at least some of the peripheral light rays entering the eye in a particular angular range, such as about 50 degrees to about 80 degrees, relative to the optical axis of the eye. In some such implementations, some of the peripheral light rays can still miss the optic and be focused onto the retina only by the cornea to form a secondary peripheral image. In other implementations, the optic prevents the formation of such a secondary image. By way of example, in some embodiments, the axial distance 27 can be smaller than about 0.8 mm, or smaller than about 0.7 mm, or smaller than about 0.6 mm. As well, the axial distance 27 can have a lower limit of about 0.01 mm.

In some embodiments, an IOL can include one or more haptics, or other fixation members, such that a free-end of the haptic or fixation member is positioned posterior to a posterior-surface of the IOL's optic. For instance, as depicted in FIG. 2B, the free-end 25A of a haptic 25 extends posteriorly from the optic 21, and in particular the end 25A is posterior to a posterior-surface 21B of the optic 21. In some implementations, the axial distance 26, i.e., a distance parallel to the optical axis 22A of the optic 21, between the optic's posterior surface 21B and the haptic's free-end 25A can be, for example, greater than about 0.4 mm, or 0.5 mm, or 0.6 mm, so as to ensure that the IOL's optic is sufficiently close to the iris for ameliorating, and preferably preventing, dysphotopsia. In some instances, the upper limit for the axial distance 26 can be about 1 mm. Such a dimension can be chosen, for example in conjunction with the dimension of the optic, such that the optic would receive all, or at least a portion, of peripheral light rays that enter the eye.

In another embodiment, an IOL can include one or more fixation members, which are adapted to contact a portion of the eye posterior to the optic. For example, as shown in FIG. 2C, an IOL 20 has a haptic 25 that contacts a cilary body at a point 25B that is posterior to the optic 21, for example posterior to a posterior-most surface of the optic 21A. The distance 28 can be an axial distance, i.e., a distance parallel to the optical axis 22 (which can be taken as the optical axis of the IOL or that of the eye), that can be at least about 0.2 mm, or at least about 0.3 mm, or at least about 0.4 mm. In some instances, the distance 28 can have an upper limit of about 1.2 mm. It is understood that where the fixation member contacts the eye can vary depending upon the configuration of the IOL. For example, haptics can be used to anchor an optic by contacting various eye features such as cilary bodies, or a portion of a capsular bag, or a region between the root of the iris and a cilary body. Other examples include the positioning of extension members in the capsular bag of an eye, as described further herein. All these potential eye contacting points are within the scope of this embodiment.

The fixation members can have a variety of structures and shapes. In some embodiments, a fixation member can be formed as one or more extension members that are coupled to a peripheral portion of the IOL's optic, and protrude there from. Such extension members can be adapted to position the optic in the capsular bag of the subject's eye, e.g., in a manner to help alleviate or prevent dysphotopsia. Decentration of an implanted IOL can be a cause of dysphotopsia, allowing peripheral light rays to miss the optic and strike the retina. Accordingly, the extension members of an IOL can be adapted to maintain centering, or positioning, of an IOL in a manner such that peripheral light rays strike the IOL to help inhibit dysphotopsia.

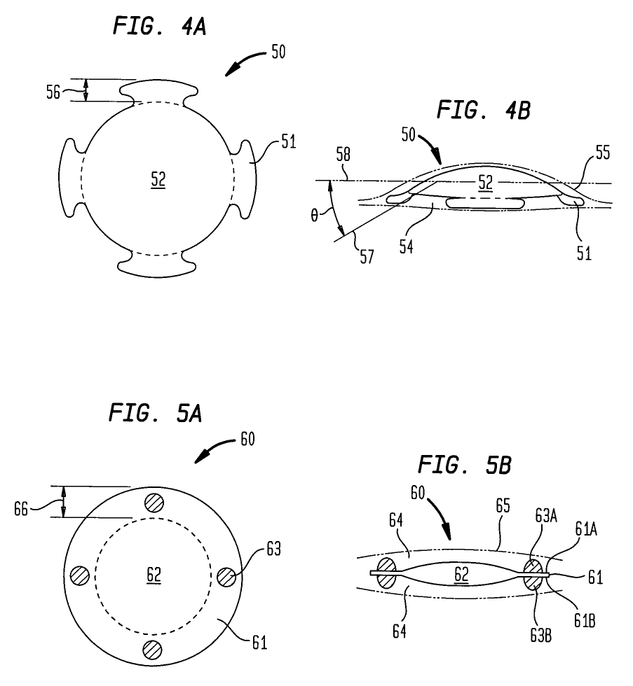

FIGS. 4A-5B provide two examples consistent with embodiments that utilize an extension member. FIG. 4A presents a schematic anterior view of an IOL 50 having four extension members 51 attached to the periphery of an optic 52. As shown in the schematic side view of FIG. 4B, the IOL 50 can be placed in a capsular bag 55 of the eye, which formerly contained a natural crystalline lens or can still contain the natural lens. The IOL 50 can be adapted to project the optic 52 towards the eye's iris, with the extensions 51 tending to be positioned posterior to the optic 52. For example, as depicted in FIG. 4B, a principal plane of the optic 58 can form an angle θ relative to a projection line 57 of the extension member to project the optic 52 towards an eye's iris. The angle can be any appropriate value from 0 to 90 degrees, or from about 0 degrees to about 45 degrees, or from about 0 degrees to about 30 degrees. Alternatively, the angle θ can range from about 5 degrees to about 45 degrees, or from about 12 degrees to about 45 degrees, or from about 15 degrees to about 45 degrees. In further alternatives, the angle θ can range from about 5 degrees to about 30 degrees, or from about 12 degrees to about 30 degrees, or from about 15 degrees to about 30 degrees.

FIGS. 5A and 5B depict another example of an IOL 60, in which an extension member is adapted as an annular structure 61 around the periphery of an optic 62. In some implementations, the annular structure 61 can have a width in a range of about 7 mm to about 10 mm. A set of protuberances 63 can be attached to the annular structure 61 for positioning the optic 62 in a capsular bag 65 as depicted in FIG. 5B, e.g., the protuberances 63 can act to suspend the remainder of the IOL when it is within the capsular bag. In this embodiment, the protuberances 63B are located on a posterior side 61B of the annular structure 61, which can act to project the optic 62 towards the iris, e.g., closer to the pupil of the eye. Protuberances 63A can also be located on an anterior surface 61A of the structure 61. In some implementations, such protuberances have a height in a range of about 0.01 mm to about 0.8 mm.

Extension members can be constructed of any appropriate material, such as those utilized in optic and/or haptic formation. In many embodiments, they are formed from polymethylmethacrylate (PMMA). It is also understood that such extensions can be formed integrally with the optic, or separately and subsequently coupled with the optic. As well, the relative sizes of the optic and the extension members can be any that make the IOL suitable for alleviating or preventing dysphotopsia and which can make the IOL suitable for implantation in a subject's eye. In some embodiments, such as that depicted in FIGS. 4A-5B, the extension members can be dimensioned to alleviate dysphotopsia, while also maintaining an extent of peripheral vision of the subject having the implanted IOL. For example, in the IOL depicted in FIGS. 5A and 5B, the width 66 of the annular structure 61 can be about 2.9 mm. The width can vary somewhat if the annular structure and/or the optic is not perfectly circular. The optic 62 can have typical dimensions, e.g., a diameter of about 6 mm. Similarly the extension members 51 of the IOL 50 depicted in FIGS. 4A and 4B can be oriented such that each has a width 56 of about 2.9 mm, though different extension members can also utilize different sizes or design configurations. Any of the extensions discussed herein can be embodied to be relatively thin, e.g., having an edge thickness less than about 0.1 mm, which can help facilitate deformation of an IOL when it is being delivered into the subject's eye.

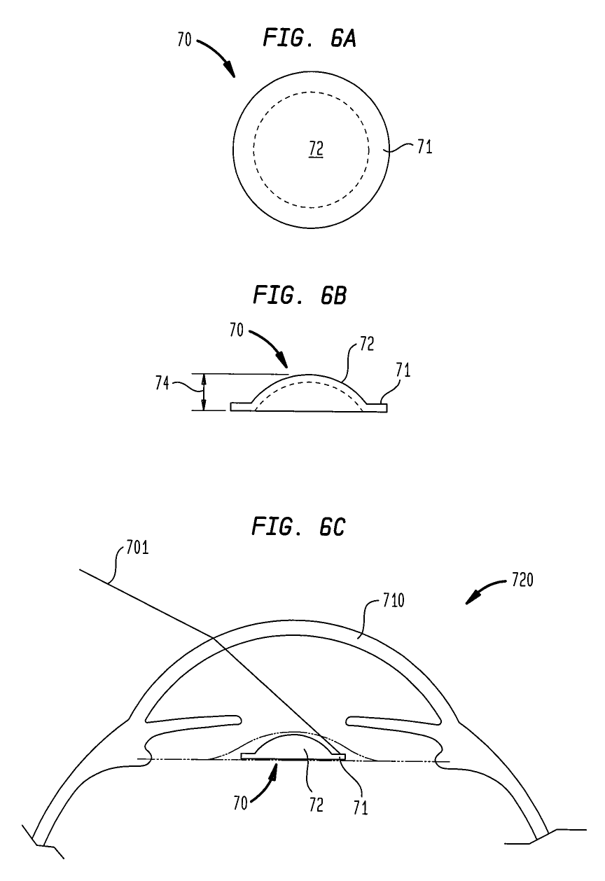

The fixation members, such as those schematically depicted in FIGS. 4A and 4B, or FIGS. 5A and 5B, can have potential advantages. For example, the protuberances can result in spaces 54, 64 between the IOL and the capsular bag, which can facilitate fluid flow in the eye. Accordingly, viscoelastic that might have been built up behind an optic can be dispelled, and excessive intraocular pressure in the eye can potentially be relieved. To enhance the removal of viscoelastic, some IOL embodiments can utilize reduced mass optics, which can enhance manipulation of the optic to allow for easier viscoelastic removal. Furthermore, extension members can potentially alleviate or prevent dysphotopsia utilizing other mechanisms beyond positioning the optic close to the eye's pupil, though such positioning can also be included. For instance, an extension member can be adapted to reduce negative dysphotopsia by intercepting light that would have otherwise bypassed an optic. As exemplified by FIGS. 6A-6C, an IOL 70 can have an extension member embodied as an annular structure attached to an optic 72 oriented toward an anterior direction, i.e., toward the cornea 710 of an eye 720. As shown in FIG. 6C, a peripheral light ray 701 can be intercepted by the annular structure 71 to help reduce the effects of negative dysphotopsia. Without the structure 71, the light ray 701 would typically miss the IOL and be imaged by the cornea on the eye's retina to form a second peripheral image. The annular structure 71 can have a variety of coatings and/or surface profiles and/or surface structures (e.g., surface textures), which can inhibit secondary image formation, or can direct some light to the retinal dark (shadow) region. For example, the structure 71 can be adapted to scatter, diffract, absorb, or refract the incident light thereon, or can provide some combination of such light altering properties. Some of these properties are discussed in a concurrently filed U.S. patent application Ser. No. entitled “Intraocular Lens with Peripheral Region Designed to Reduce Negative Dysphotopsia,” bearing attorney docket number 2817, which is hereby incorporated by reference in its entirety herein.

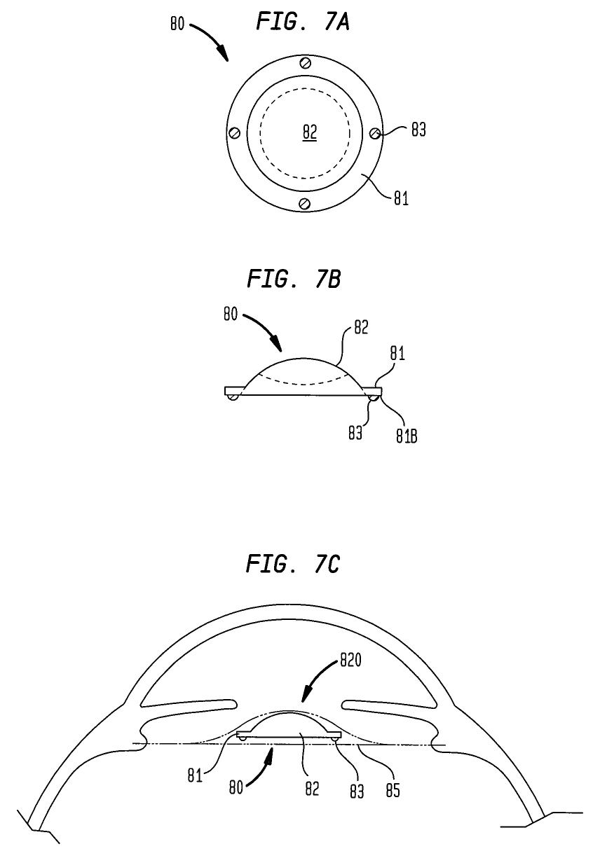

FIGS. 7A-7C depict another exemplary embodiment of an IOL utilizing an extension member as a fixation member. As depicted in FIGS. 7A and 7B, an IOL 80 includes an optic 82 having an annular structure 81 attached to the optic's peripheral portion. The annular structure 81 also includes protuberances 83 on a posterior surface 8 1B thereof. As shown in FIG. 7C, the IOL 80 can be positioned within a capsular bag 85. The protuberances 83 and the position of the optic 82 relative to the annular structure 81 can each act to project the optic 82 closer to the pupil 820 of the eye, which can help alleviate or prevent dysphotopsia.

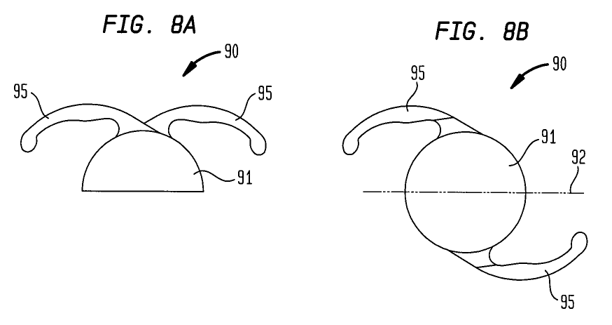

Other exemplary features of an IOL, which embody aspects of the present application, are illustrated in FIGS. 8A and 8B. For example, in many embodiments, IOLs as utilized herein can generally be configured as deformable structures that can be delivered in a compact manner to an implantation site. As one example depicted in FIG. 8A, an IOL 90 can be folded in half along a dimension 92 of the optic 91 for insertion in a direction perpendicular to an incision. Accordingly, the size of the incision can be about half as large as needed if the IOL was not folded. Upon delivery, such IOLs can be adapted to unfold to an open configuration, as exemplified in FIG. 8B, and can be positioned for fixation in the subject's eye. Typically, it can be desirable to make such IOLs as small as effectively possible to minimize the size of the incision needed to deliver the IOL. It is understood that other deformable configurations are also possible, such as deforming the IOL to fit in a tubular delivery structure.

Further exemplary features of IOLs include the use of optics that provides multiple optical focusing powers. By way of one embodiment, a diffractive structure can be disposed on an anterior surface (or a posterior surface or both surfaces) of the optic such that the optic would provide not only a far-focus optical power (e.g., in a range of about −15 D to about 34 D) but also a near-focus optical power, e.g., in a range of about 1 D to about 4 D. The optic's diffractive structure can be configured to include a plurality of diffractive zones that are separated from one another by a plurality of steps that exhibit a decreasing height as a function of increasing distance from the optical axis OA—though in other embodiments the step heights can be uniform. In other words, in this embodiment, the step heights at the boundaries of the diffractive zones are “apodized” so as to modify the fraction of optical energy diffracted into the near and far foci as a function of aperture size (e.g., as the aperture size increases, more of the light energy is diffracted into the far focus). By way of example, the step height at each zone boundary can be defined in accordance with the following relation:

$$\text{Step height} = \frac{λ}{a ( {n}_{2} - {n}_{1} )} {f}_{apodize} \qquad Equation ( 1 )$$

wherein

λ denotes a design wavelength (e.g., 550 nm),

a denotes a parameter that can be adjusted to control diffraction efficiency associated with various orders, e.g., a can be selected to be 1.9;

n₂ denotes the index of refraction of the lens material,

n₁ denotes the refractive index of a medium in which the lens is placed, and

ƒ_{apodize} represents a scaling function whose value decreases as a function of increasing radial distance from the intersection of the optical axis with the anterior surface of the lens. By way of example, the scaling function ƒ_{apodize} can be defined by the following relation:

$${f}_{apodize} = 1 - {( \frac{{r}_{i}}{{r}_{out}} )}^{3} . \qquad Equation ( 2 )$$

wherein

rᵢ denotes the radial distance of the i^{th} zone,

rₒᵤₜ denotes the outer radius of the last bifocal diffractive zone. Other apodization scaling functions can also be employed, such as those disclosed in a co-pending patent application entitled “Apodized Aspheric Diffractive Lenses,” filed Dec. 1, 2004 and having a Ser. No. 11/000770, which is herein incorporated by reference.

In this exemplary embodiment, the diffractive zones are in the form of annular regions, where the radial location of a zone boundary (rᵢ) is defined in accordance with the following relation:

rᵢ²=(2i+1)λƒ Equation (3)

wherein

i denotes the zone number (i=0 denotes the central zone),

rᵢ denotes the radial location of the ith zone,

λ denotes the design wavelength, and

ƒ denotes an add power.

It is understood that various embodiments of IOLs can utilize or contain features described in other embodiments, and that the scope of the present invention is not necessarily limited to the explicitly described embodiments herein. For instance, features of IOLs using haptics as fixation members can also be used in embodiments that utilize extension members as fixation members. For example, embodiments which describe the axial distance between an anterior-most portion of an optic and the tip of the iris; or the distance between an end point of a fixation member and a posterior surface of the optic of an IOL, or the distance of position of an anterior-most portion of an optic relative to where a portion of a fixation member contacts an eye can be applied to IOL with extensions as fixation members, as opposed to haptics. In one particular example, the distance 74 between the edge of an annular structure 71 and an anterior-most surface of an optic 72, as depicted in FIG. 6B, can be at least a particular distance of about 0 mm to about 1.2 mm, or about 0.2 mm to about 1.2 mm. Accordingly, it is understood that embodiments consistent with the present invention can utilize any number of the features described herein with respect to other embodiments.

IOLs according to the teachings of the invention, such as the above embodiments, can be employed in methods of correcting vision, e.g., to replace a clouded natural lens. For example, in cataract surgery, a clouded natural lens can be removed and replaced with an IOL. By way of example, an incision can be made in the cornea, e.g., via a diamond blade, to allow other instruments to enter the eye. Subsequently, the anterior lens capsule can be accessed via that incision to be cut in a circular fashion and removed from the eye. A probe can then be inserted through the corneal incision to break up the natural lens via ultrasound or other techniques. The lens fragments can be subsequently aspirated. An IOL according to the teachings of an aspect of the invention, which can include an optic and at least one fixation member projecting the optic toward the pupil, can be implanted into the eye to correct vision while inhibiting dysphotopsia. For example, forceps can be employed to place the IOL in a folded state in the original lens capsule. Upon insertion, the IOL can unfold and its haptics can anchor it within the capsular bag.

In some cases, the IOL is implanted into the eye by utilizing an injector system rather than employing forceps insertion. For example, an injection handpiece having a nozzle adapted for insertion through a small incision into the eye can be used. The IOL can be pushed through the nozzle bore to be delivered to the capsular bag in a folded, twisted, or otherwise compressed state. The use of such an injector system can be advantageous as it allows implanting the IOL through a small incision into the eye, and further minimizes the handling of the IOL by the medical professional. By way of example, U.S. Pat. No. 7,156,854 entitled “Lens Delivery System,” which is herein incorporated by reference, discloses an IOL injector system. The IOLs according to the embodiments of the invention, are preferably designed to inhibit dysphotopsia while ensuring that their shapes and sizes allow them to be inserted into the eye via injector systems through small incisions.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments in any suitable combination. Such modifications and variations are intended to be included within the scope of the present invention. As well, one skilled in the art will appreciate further features and advantages of the invention based on the above-described embodiments. Accordingly, the invention is not to be limited by what has been particularly shown and described, except as indicated by the appended claims.

What is claimed is:

1. An intraocular lens (IOL), comprising:
an optic for implantation in a subject's eye; and
at least one fixation member coupled to said optic for anchoring said optic in the subject's eye, said at least one fixation member adapted to position said optic sufficiently close to the iris to inhibit dysphotopsia.

2. The IOL of claim 1, wherein said at least one fixation member is adapted to project said optic towards the iris.

3. The IOL of claim 1, wherein said at least one fixation member is adapted to position said optic to intercept peripheral light rays entering a pupil of the subject's eye at angles from about 50 degrees to about 80 degrees relative to the eye's visual axis.

4. The IOL of claim 1, wherein said at least one fixation member comprises an arm extending posteriorly from said optic and forming an angle in a range of about 5 degrees to about 45 degrees relative to a principal plane of said optic.

5. The IOL of claim 1, wherein said at least one fixation member is adapted to position said optic such that an anterior-most portion of said optic and a tip of an iris are separated by an axial distance of less than 0.8 mm apart.

6. The IOL of claim 1, wherein said at least one fixation member is adapted to contact a portion of the eye posterior to an anterior-most portion of said optic.

7. The IOL of claim 1, wherein said at least one fixation member comprises at least one extension member coupled to a peripheral portion of said optic, said at least one extension member adapted to position said optic in a capsular bag of the subject's eye.

8. The IOL of claim 7, wherein said at least one extension member includes a line of projection forming an angle with a principal plane of the optic in a range from about 5 degrees to about 45 degrees.

9. The IOL of claim 7, wherein said at least one extension member comprises an annular structure coupled to said peripheral portion of said optic.

10. The IOL of claim 7, wherein said at least one extension member includes at least one protuberance extending from a surface of said at least one extension member, said protuberance adapted to contact the capsular bag of the subject's eye.

11. The IOL of claim 10, wherein said protuberance is adapted to contact at least one of an anterior surface and a posterior surface of the capsular bag.

12. The IOL of claim 1, wherein said at least one fixation member comprises at least one haptic coupled to said optic.

13. The IOL of claim 1, wherein said at least one fixation member is adapted to implant the said optic posterior to an iris of the subject's eye.

14. A method of inhibiting dysphotopsia in a patient's eye, comprising:
implanting the IOL of claim 1 in the patient's eye.

15. An intraocular lens (IOL), comprising:
an optic for implantation in a subject's eye; and
at least one haptic coupled to said optic, said at least one haptic having a free-end positioned posterior to a posterior-surface of said optic.

16. The IOL of claim 15, wherein said at least one haptic is adapted such that said free-end and said posterior-surface are an axial distance of at least 0.4 mm apart.

17. The IOL of claim 15, wherein said at least one haptic is adapted to position the said optic to intercept peripheral light rays entering a pupil of the subject's eye at angles from about 50 degrees to about 80 degrees relative to an optical axis of the subject's eye.

18. The IOL of claim 15, wherein said at least one haptic is adapted to position the said optic such that an anterior-most portion of said optic and a tip of the iris are an axial distance of less than 0.8 mm apart.

19. The IOL of claim 15, wherein said at least one haptic is adapted to contact a portion of the eye posterior to an anterior-most portion of said optic.

20. A method of inhibiting dysphotopsia in a patient's eye, comprising:
implanting the IOL of claim 15 in the patient's eye.

21. An intraocular lens (IOL), comprising:
an optic for implantation posterior to an iris of a subject's eye; and
at least one fixation member coupled to said optic, said at least one fixation member adapted to position said optic such that an anterior-most portion of said optic and a tip of the iris are an axial distance of less than 0.8 mm apart.

22. The IOL of claim 21, wherein said at least one fixation member is adapted to position said optic to intercept peripheral light rays entering a pupil of the subject's eye at angles from about 50 degrees to about 80 degrees relative to an optical axis of the subject's eye.

23. The IOL of claim 21, wherein said at least one fixation member is adapted to contact a portion of the eye posterior to an anterior-most portion of said optic.

24. The IOL of claim 21, wherein said at least one fixation member comprises at least one extension member coupled to a peripheral portion of said optic, said at least one extension member adapted to position said optic in a capsular bag of the subject's eye.

25. The IOL of claim 24, wherein said at least one extension member comprises an annular structure coupled to said peripheral portion of said optic.

26. The IOL of claim 24, wherein said at least one extension member includes at least one protuberance extending from a surface of said at least one extension member, said protuberance adapted to contact the capsular bag of the subject's eye.

27. The IOL of claim 26, wherein said protuberance is adapted to contact at least one of an anterior surface and a posterior surface of the capsular bag.

28. The IOL of claim 21, wherein said at least one fixation member comprises at least one haptic coupled to said optic.

29. A method of inhibiting dysphotopsia in a patient having an intraocular lens (IOL), comprising:
positioning an anterior surface of the IOL in a posterior chamber of a patient's eye close enough to an iris to inhibit dysphotopsia.

30. The method of claim 29, wherein positioning the anterior surface includes positioning the anterior surface of the IOL such that peripheral light rays that enter a pupil intercept the anterior surface.

31. The method of claim 30, wherein said peripheral light rays intercepting the anterior surface are directed to hinder the formation of a secondary image on a retina of the patient's eye.

32. The method of claim 30, wherein said peripheral light rays enter said pupil at angles between about 50 degrees and about 80 degrees relative to an optical axis of the patient's eye.

33. The method of claim 29, wherein the step of positioning the anterior surface includes positioning the anterior surface an axial distance of less than about 0.8 mm from a tip of an iris of a patient's eye.

34. The method of claim 29, wherein the step of positioning the anterior surface includes positioning an anterior surface of an optic of the IOL.
